(19) United States
(12) Patent Application Publication
(10) Pub. No.: US 2023/0277709 A1
(43) Pub. Date: Sep. 7, 2023

(54) CONTROL SYSTEM, CONTROL METHOD, AND RECORDING MEDIUM

(71) Applicant: Panasonic Intellectual Property Management Co., Ltd., Osaka (JP)

(72) Inventor: Katsuhiko HIRAMATSU, Osaka (JP)

(21) Appl. No.: 18/019,296

(22) PCT Filed: Aug. 11, 2021
(86) PCT No.: PCT/JP2021/029593

§ 371 Date: Feb. 2, 2023

(30) Foreign Application Priority Data
Aug. 31, 2020 (JP) 2020-145365

(51) Int. Cl. A61L 9/015

(52) U.S. Cl. CPC A61L 9/015; A61L 2209/111; A61L 2209/16

(57) ABSTRACT

This control system includes a ventilation device which performs exchange between gas inside a room including an infectious object and gas outside the room, to discharge and remove the infectious object; a supply device which supplies an inactivating substance, for inactivating the infectious object, to inside the room to inactivate and remove the infectious object; and a control device which controls the ventilation device and the supply device. The control device switches between a first mode and a second mode which is different from the first mode. The first mode is for performing at least one of a first operation whereby, on the occasion of increasing the removal capability of one of the ventilation device and the supply device, the removal capability of the other is decreased, and a second operation whereby, on the occasion of decreasing the removal efficiency of one of the ventilation device and the supply device, the removal efficiency of the other is increased.

### TECHNICAL FIELD

The present disclosure relates to a control system that controls various devices for preventing transmission of an infectious material.

### BACKGROUND ART

In recent years, various technological developments have been made to prevent transmission of infectious substances (also referred to as infectious materials) such as pathogenic virus to persons. For example, Patent Literature (PTL) 1 discloses a system for monitoring whether object persons belonging to a predetermined facility such as a hospital perform hand antisepsis.

### CITATION LIST

### Patent Literature

[PTL 1] Japanese Unexamined Patent Application Publication No. 2019-096145

### Non Patent Literature

[NPL 1] S. N. Rudnick et al. Indoor Air; 13: 237-245(2003)
[NPL 2] Hui Dai et al. medRxiv; 2020.04.21.20072397 (2020)
[NPL 3] E. C. Riley et al. American Journal of Epidemiology; 107, Issue 5: 421-432(1978)

### SUMMARY OF INVENTION

### Technical Problem

Meanwhile, in order to prevent transmission of an infectious material to persons, besides inactivation removal by inactivating the infectious material through antisepsis or the like disclosed in PTL 1, discharge removal by discharging the infectious material outside a room is also known as an effective method.

In view of the above, an object of the present disclosure is to provide a control system or the like that can more effectively prevent transmission of an infectious material to persons.

### Solution to Problem

A control system according to one aspect of the present disclosure includes: a ventilation device that replaces indoor-space aft containing an infectious material with outdoor-space aft to perform discharge removal of the infectious material; a supply device that supplies an inactivation agent for inactivating the infectious material to an indoor space to perform inactivation removal of the infectious material; and a control device that controls the ventilation device and the supply device, in which the control device switches between a first mode and a second mode. The first mode is a mode in which at least one of the following is performed: a first operation that decreases a removal capability of one of the ventilation device and the supply device when a removal capability of an other of the ventilation device and the supply device is increased; or a second operation that increases the removal capability of the one of the ventilation device and the supply device when the removal capability of the other of the ventilation device and the supply device is decreased. The second mode is different from the first mode.

Moreover, a control method according to one aspect of the present disclosure is a control method of controlling a ventilation device and a supply device. The ventilation device replaces indoor-space air containing an infectious material with outdoor-space air to perform discharge removal of the infectious material. The supply device supplies an inactivation agent for inactivating the infectious material to an indoor space to perform inactivation removal of the infectious material. The control method includes: a first step of performing at least one of the following: a first operation that decreases a removal capability of the supply device when a removal capability of the ventilation device is increased; or a second operation that increases the removal capability of the supply device when the removal capability of the ventilation device is decreased; and a second step of performing an operation different from that of the first step.

Moreover, one aspect of the present disclosure can be implemented as a program for causing a computer to execute the above control method. Alternatively, one aspect of the present disclosure also can be implemented as a non-transitory computer-readable medium storing the program.

### Advantageous Effects of Invention

According to the present disclosure, it is possible to more effectively prevent transmission of an infectious material to persons.

### BRIEF DESCRIPTION OF DRAWINGS

FIG. 1 is an overview diagram illustrating an example of use of a control system according to an embodiment.

FIG. 2 is a block diagram illustrating the functional configuration of the control system according to the embodiment.

FIG. 3A is a flowchart illustrating an exemplary operation including the first operation of the control system according to the embodiment.

FIG. 3B is a flowchart illustrating an exemplary operation including the second operation of the control system according to the embodiment.

FIG. 4 is a diagram illustrating the first specific operation according to the present embodiment.

FIG. 5 is a diagram illustrating the second specific operation according to the present embodiment.

FIG. 6 is a diagram illustrating the multiplication rates of the typical viruses.

FIG. 7 is the first graph illustrating a transition of the infection probability with respect to the elapsed time,

FIG. 8 is the second graph illustrating a transition of the infection probability with respect to the elapsed time.

FIG. 9 is a diagram illustrating the third specific operation according to the present embodiment.

FIG. 10 is a graph illustrating the relationship between the elapsed time and the ventilation air volume.

FIG. 11 is a block diagram illustrating the functional configuration of the control device including a reservation management device according to the embodiment.

FIG. 12 is a flow chart illustrating the operation related to a proposal of a condition of use, of the reservation management system for use of indoor space according to the present embodiment.

### DESCRIPTION OF EMBODIMENT

A control system or the like according to an embodiment of the present disclosure is described below in detail with reference to the drawings. Note that the embodiment described below shows a specific example of the present disclosure. The numerical values, shapes, materials, elements, the arrangement and connection of the elements, steps, and the order of processing the steps, for instance, described in the following embodiment are examples, and thus are not intended to limit the present disclosure. Among the elements in the following embodiment, elements not recited in any of the independent claims are described as arbitrary elements.

Note that the diagrams are schematic diagrams, and do not necessarily provide strictly accurate illustration. For example, the scale of each diagram is not necessarily the same. In the drawings, the same numeral is given to a substantially same configuration, and a redundant description thereof may be omitted or simplified.

### Embodiment

(Outline)

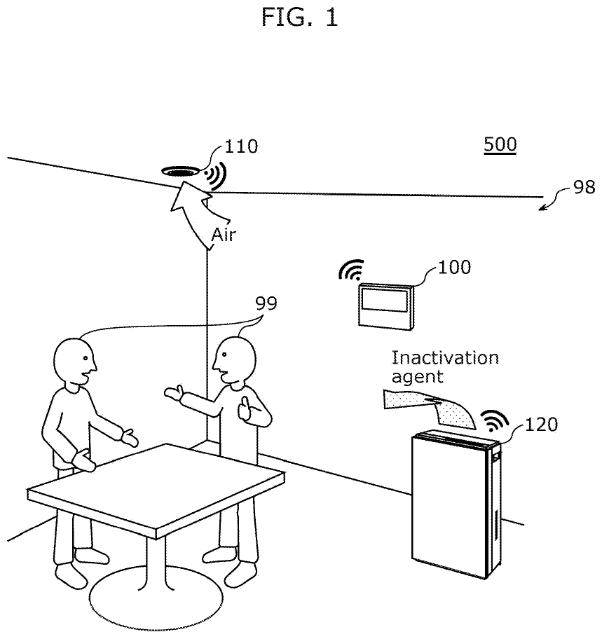

First, the outline of a control system according to an embodiment is described with reference to FIG. 1. FIG. 1 is an overview diagram illustrating an example of use of the control system according to the embodiment. FIG. 1 shows indoor space 98 in which devices related to control system 500 are placed. Note that indoor space 98 described here means a space semi-closed by wall units, a floor unit, a ceiling unit, openable doors and windows for defining the inside and outside of indoor space 98, etc. Accordingly, indoor space 98 may be a space inside a room as shown in FIG. 1, or a whole space inside a building including rooms, for example.

As shown in FIG. 1, control system 500 includes ventilation device 110, supply device 120, and control device 100.

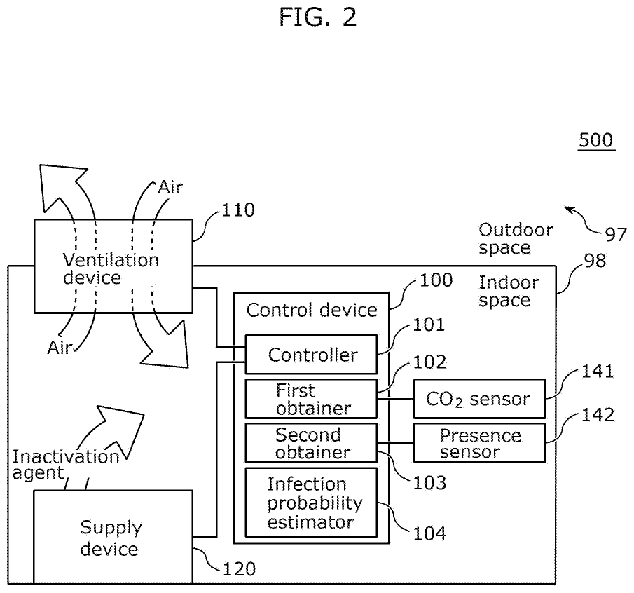

Ventilation device 110 replaces indoor-space 98 air with outdoor-space 97 air (see FIG. 2 as described later). In other words, ventilation device 110 performs ventilation. In the present embodiment, ventilation device 110 is placed on the ceiling unit in indoor space 98, and sucks out indoor-space 98 air. The indoor-space 98 air may contain infectious material.

Here, the infectious material has a lot of types classified as, for example, bacteria, virus, and particles such as nucleic acid and protein. In some of the types, to prevent transmission is required since the infectious material is transmitted from one person to another. In particular, as shown in FIG. 1, for example, when persons 99 talk with each other in same indoor space 98, the infectious material with which one person 99 is infected is splashed toward other person 99 through the space, and thus infection is more likely to spread. In particular, when infected person 99 does not take proper countermeasures due to unawareness or the like, explosive spread of infection may occur.

Such an infectious material is relatively light in weight in many cases. It is known that the infectious material remains in indoor space 98 for a long time by, for example, floating in indoor space 98. For example, the infectious material is discharged to outdoor space 97 by replacing indoor-space 98 air containing the infectious material with outdoor-space 97 air using ventilation device 110, and thus it is possible to prevent transmission to persons 99. Hereinafter, such removal of the infectious material from indoor space 98 by discharging the infectious material outside a system, e.g., outdoor space 97, is referred to as discharge removal.

Ventilation device 110 replaces air by blowing indoor-space 98 air toward outdoor space 97 using a fan or the like and introducing outdoor-space 97 air into indoor space 98. The present disclosure shows, as an example, a so-called type III ventilation system in which air is replaced by only blowing indoor-space 98 air toward outdoor space 97 such that negative-pressure indoor space 98 automatically sucks in outdoor-space 97 air. In the present disclosure, a type I ventilation system and a type II ventilation system are also possible, and the ventilation system and the device configuration related to the ventilation are not particularly limited. Accordingly, any other ventilation device is also possible as long as indoor-space 98 air is replaced with outdoor-space 97 air.

Supply device 120 is placed on the floor unit in indoor space 98, and supplies an inactivation agent for inactivating the infectious material in indoor space 98. The inactivation agent is, for example, alcohol such as ethanol, an invert soap such as benzalkonium chloride, or hypochlorite, which exerts an inactivation effect by collapsing the cell membrane of bacteria and denaturing the macromolecule.

For example, supply device 120 vaporizes hypochlorite solution made by electrolysis of salt water using a fan or a water-absorbing filter, and sprays the hypochlorite into indoor space 98 as the inactivation agent. The sprayed hypochlorite kills (inactivates) the infectious material by coming into contact with the infectious material present in the space, collapsing its structure such as a cell membrane or an outer protein shell, and denaturing its nucleic acid, enzyme protein, and the like. To remove an active infectious material from indoor space 98 by inactivating the active infectious material in this manner is referred to as inactivation removal.

Note that supply device 120 is not limited to the above configuration. For example, supply device 120 produces the same effect even when the device is configured to suck indoor-space 98 air into the body of the device and release the sucked air after forcing the sucked air to contact with the inactivation agent. In this case, to “supply an inactivation agent to indoor space 98” means the configuration in which at least air in indoor space 98 is forced to contact with the inactivation agent. In other words, the concept of supply of the inactivation agent using supply device 120 includes that at least air in indoor space 98 is forced to contact with the inactivation agent.

In the present embodiment, supply device 120 is configured to force indoor-space 98 air to contact with the inactivation agent by spraying the inactivation agent and also force the infectious material attached to an object such as the wall unit, the floor unit, and furniture or appliance in indoor space 98 to contact with the inactivation agent. Accordingly, the configuration according to the present embodiment is more effective in preventing transmission of the infectious material than the above exemplary configuration in which only indoor-space 98 air is forced to contact with the inactivation agent.

Control device 100 switches an operational mode by controlling ventilation device 110 and supply device 120, to appropriately perform the discharge removal and the inactivation removal. For example, control device 100 controls ventilation device 110 and supply device 120 by wirelessly communicating with these devices. As one example, control device 100 is placed on the wall unit, and has a control panel. Control device 100 includes a processor and a storage. Control device 100 controls ventilation device 110 and supply device 120 using a predetermined control algorithm by causing the processor to execute a program stored in the storage. The details of the predetermined control algorithm will be described later.

Note that the control panel of control device 100 is, for example, a device that receives an input from person 99 present in indoor space 98. For example, this input is used to provide a variable parameter to an algorithm for controlling ventilation device 110 and supply device 120.

The foregoing describes an example in which control device 100 is placed in indoor space 98, but control device 100 need not be a single device as described above. For example, control device 100 may be included in ventilation device 110 or supply device 120, or may be established away from indoor space 98 using a cloud server, an edge server, or the like. In this case, control device 100 may be communicably connected to ventilation device 110 and supply device 120 via a wide area communication network such as the Internet, a local area network in a building, or the like.

Next, with a focus on control device 100, the detailed structure of each part is described with reference to FIG. 2. FIG. 2 is a block diagram illustrating the functional configuration of the control system according to the embodiment. As shown in FIG. 2, control device 100 according to the present embodiment includes controller 101, first obtainer 102, second obtainer 103, and infection probability estimator 104. Controller 101 is a functional unit that controls ventilation device 110 and supply device 120. Controller 101 is implemented by causing a processor and a storage to execute a program for performing a predetermined process.

Controller 101 determines the removal capability of ventilation device 110 to remove the infectious material in accordance with a predetermined control algorithm. The removal capability described here means a removal amount of the infectious material removed by the discharge removal. In the discharge removal of the infectious material, the infectious material floating in air is a target of the removal. The removal amount depends on the dispersity of the infectious material into air and the volume of discharged air (i.e., a ventilation air volume). For example, when it is assumed that the infectious material instantly disperses into air uniformly, the removal amount is simply proportional to the ventilation air volume. In the present disclosure, the above assumption is employed for the sake of simplifying the calculation. However, for example, the dispersion rate of the infectious material and the placement position of ventilation device 110 may be additionally taken into account to perform the calculation. Note that the ventilation air volume means a volume of indoor-space 98 air replaced with outdoor-space 97 air per unit time.

In this manner, control device 100 generates a control signal for specifying a ventilation air volume of ventilation device 110, and sends the control signal to ventilation device 110. Ventilation device 110 receives the control signal, and operates according to the control signal.

Controller 101 also determines the removal capability of supply device 120 to remove the infectious material in accordance with a predetermined control algorithm. The removal capability described here means an amount of the infectious material removed using the inactivation removal. In the inactivation removal of the infectious material, the infectious material floating in air and the infectious material attached to an object are targets of the removal. The removal amount depends on not only the dispersity of the inactivation agent into air and the volume of supplied inactivation agent, but also the ratio of contact in air between the infectious material and the inactivation agent and various conditions related to reactions such as the reaction rate of each reaction from contact until inactivation. For example, when it is assumed that the inactivation agent instantly disperses into air uniformly and the various conditions related to reactions are always constant, the removal amount is simply proportional to the volume of supplied inactivation agent (spray volume). In the present disclosure, the above assumption is employed for the sake of simplifying the calculation. However, for example, the dispersion rate of the inactivation agent, the placement position of supply device 120, the various conditions related to reactions, etc. may be additionally taken into account to perform the calculation.

In this manner, control device 100 generates a control signal for specifying a volume of supplied inactivation agent of supply device 120, and sends the control signal to supply device 120. Supply device 120 receives the control signal, and operates according to the control signal.

First obtainer 102 is a communication module for obtaining a CO₂ concentration in indoor space 98 from CO₂ sensor 141 that is a sensor for measuring the CO₂ concentration in indoor space 98. First obtainer 102 is communicably connected to CO₂ sensor 141. The obtained CO₂ concentration in indoor space 98 is used in a step of the predetermined control algorithm as described later, and thus will be described later along with the predetermined control algorithm.

Second obtainer 103 is a communication module for obtaining presence information regarding whether a person is present in indoor space 98 from presence sensor 142 that is a sensor for detecting whether a person is present in indoor space 98. Second obtainer 103 is communicably connected to presence sensor 142. The obtained presence information regarding whether a person is present in indoor space 98 is used in a step of the predetermined control algorithm as described later, and thus will be described later along with the predetermined control algorithm.

Infection probability estimator 104 is a functional unit for calculating, based on estimation, an infection probability in transmission of the infectious material to person 99. Infection probability estimator 104 is implemented by causing a processor and a storage to execute a program for performing a predetermined process. For example, infection probability estimator 104 receives various parameters contributing to the estimation which are inputted to the control panel or the like, and calculates the infection probability of person 99 in indoor space 98 using the parameters. The calculated infection probability is used in a step of the predetermined control algorithm as described later, and thus will be described later along with the predetermined control algorithm,

(Control Algorithm)

The following describes a predetermined control algorithm for control device 100 to control ventilation device 110 and supply device 120. In the present embodiment, a capability of removing the infectious material (also referred to as the removal capability) is kept at a certain level in total, and a balance of the performance between ventilation device 110 and supply device 120 to achieve a constant total removal capability is changed as needed. In this manner, transmission of the infectious material to persons is more effectively prevented. The control algorithm as described above is used to determine the performance of ventilation device 110 and the performance of supply device 120,

First, the removal capability of ventilation device 110 is discussed. The concentration of the infectious material is changed by replacement of indoor air with outdoor air through ventilation, according to the following equation (1):

[Math. 1]

CₒQdt−C(t)Qdt=VdC (1)

where t denotes the elapsed time [h], C(t) denotes the concentration of the infectious material in indoor space 98 at time t [mg/m³], Cₒ denotes the concentration of the infectious material in outdoor space 97 [mg/m³], and V denotes the size of indoor space 98 [m³]. Note that Cₒ is assumed to be constant since the infectious material is attenuated infinitely even when the infectious material is discharged from indoor space 98. The above equation (1) is rearranged to obtain the following differential equation (2).

$$\begin{matrix}[ Math . 2 ] & \\ \frac{V}{Q} \frac{dC}{dt} = {C}_{o} - C ( t ) & ( 2 )\end{matrix}$$

Given that C(t) at t=0 is C_{S}, the above equation (2) is transformed into the following equation (3).

$$\begin{matrix}[ Math . 3 ] & \\ C ( t ) = {C}_{o} + ( {C}_{s} - {C}_{o} ) {e}^{- \frac{Q}{V} t} & ( 3 )\end{matrix}$$

Here, the removal capability of ventilation device 110 can be regarded as the amount of change in concentration of the infectious material with respect to the elapsed time. Note that a difference in concentration of the infectious material between the indoor space and the outdoor space contributes to this numerical value. Accordingly, when the data is normalized with respect to the difference in concentration, residual rate X₁(t) which is the opposite side of the removal capability of ventilation device 110 is expressed by the following equation (4):

$$\begin{matrix}[ Math . 4 ] & \\ {X}_{1} ( t ) = {e}^{- \frac{Q}{V} t} & ( 4 )\end{matrix}$$

where Q denotes the replacement air volume per unit time (here, one hour), i.e., ventilation air volume [m³/h]. Accordingly, Q×t/V in the above equation (4) denotes the number of ventilations in the space of size V.

On the other hand, the removal capability of supply device 120 can be regarded as the amount of accumulated partial infectious material inactivated by the inactivation agent sprayed during the elapsed time. On the opposite side of the same coin, the removal capability of supply device 120 is formulated as a value obtained by raising a ratio of the residual active infectious material after partial inactivation for each unit time relative to the original active infectious material, to the power of the elapsed time. In other words, residual rate X₂(t) which is the opposite side of the removal capability of supply device 120 is expressed by the following equation (5):

[Math. 5]

X₂(t)=β^{t} (5)

where β denotes the residual rate of the infectious material per unit time. Note that β is greater than 0 and less than 1 (0<β<1). Here, for example, when the inactivation agent is sprayed under predetermined conditions and it is assumed that 99.99% of the infectious material is removed 12 hours later, the equation (5) is X₂(12)=β¹²=0.0001. In this case, β is 0.464. In other words, under the above exemplary conditions, it is found that 53.6% of the infectious material is removed per unit time by the inactivation agent using the inactivation removal.

Here, residual rate X₁(t) and residual rate X₂(t) are each derived from different removal of the infectious material which bases an independent effect. Accordingly, when ventilation device 110 and supply device 120 operate simultaneously, the total removal capability for the infectious material is expressed using the residual rate of the infectious material as Xₜ(t) by the following equation (6).

[Math. 6]

Xₜ(t)=X₁(t)×X₂(t) (6)

In other words, the following equation (7) is obtained from the above equations (4) and (5).

$$\begin{matrix}[ Math . 7 ] & \\ {X}_{t} ( t ) = {e}^{- \frac{Q}{V} t} \times {β}^{t} & ( 7 )\end{matrix}$$

The above equation (7) is transformed into the following equations (8) and (9) by rearranging the constants.

$$\begin{matrix}[ Math . 8 ] & \\ \frac{{Q}_{t}}{V} = \frac{Q}{V} - ln β & ( 8 )\end{matrix}$$

$$\begin{matrix}[ Math . 9 ] & \\ {Q}_{t} = Q - V ln β & ( 9 )\end{matrix}$$

Note that, in the above equations (8) and (9), Qₜ denotes the ventilation air volume of when it is assumed that the total removal capability of both ventilation device 110 and supply device 120 is implemented by only ventilation (i.e., the equivalent ventilation aft volume) [m³/h], and corresponds to the value obtained by multiplying the natural logarithm of Xₜ(t) by −(1/t),

In order to keep the effectiveness in the removal of the infectious material at a certain level, it is needed to keep the numerical value calculated by the above equation (8) or (9) at least at a certain level. In other words, as long as the numerical value calculated by the above equation (8) is kept at least at a certain level, the effectiveness in the removal of the infectious material can be kept at a certain level even when the removal capability of one of ventilation device 110 and supply device 120 is reduced. In other words, control device 100 can perform a control mode (the first mode) in which at least one of the following is performed according to the above equation (8): the first operation that decreases the removal capability of one of ventilation device 110 and supply device 120 when the removal capability of the other of ventilation device 110 and supply device 120 is increased; or the second operation that increases the removal capability of one of ventilation device 110 and supply device 120 when the removal capability of the other of ventilation device 110 and supply device 120 is decreased.

Moreover, control device 100 can combine the above control mode with a uni-control mode (one example of the second mode) in which each of the devices operates at a constant removal capability or a uni-control mode (another example of the second mode) in which either one of the devices operates at a constant removal capability. In the case where either one of the devices is controlled to operate at a constant removal capability, the removal capability of one of the devices may be kept constant even when the removal capability of the other of the devices increases, and the total removal capability may be kept constant according to the above equation (8) only when the removal capability of the other of the devices decreases. In other words, also in the second mode, the operational control according to the above equation (8) may be performed,

(Exemplary Operation)

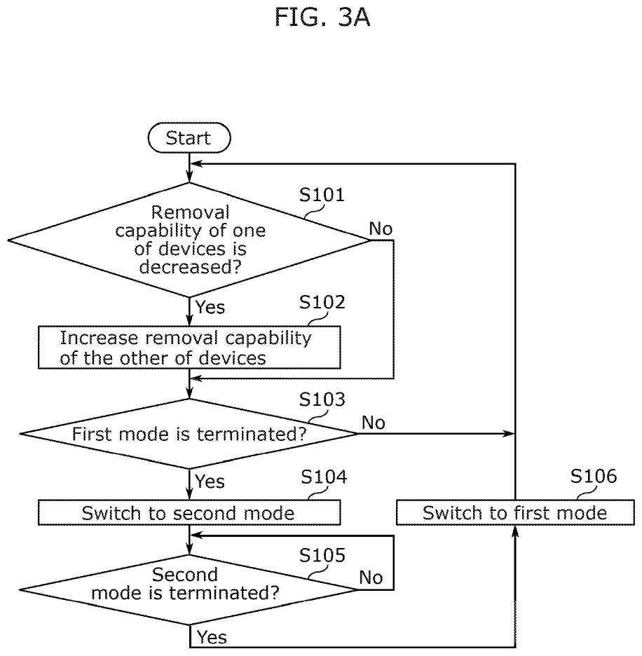

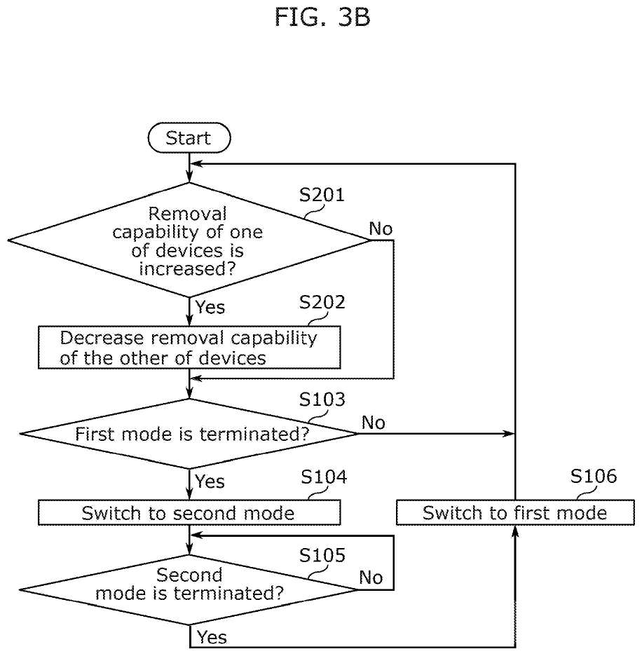

The operation of control system 500 configured as described above is described with reference to FIG. 3A and FIG. 33. FIG. 3A is a flowchart illustrating an exemplary operation including the first operation of the control system according to the embodiment. FIG. 33 is a flowchart illustrating an exemplary operation including the second operation of the control system according to the embodiment. The exemplary operation of FIG. 3A and the exemplary operation of FIG. 313 are different in the steps according to the first operation and the steps according to the second operation, and the same operation is performed in the other steps. Accordingly, in the following description, the same numeral is given to a duplicated step, and a description thereof may be omitted.

As shown in FIG. 3A, control system 500 according to the present embodiment performs the first mode first. In the first mode, as described above, control is performed such that the removal capability of one of ventilation device 110 and supply device 120 is increased when the removal capability of the other of ventilation device 110 and supply device 120 decreases. In control system 500, when ventilation device 110 and supply device 120 are controlled, for example, it may be necessary to decrease the removal capability of one of the devices due to other control factors. In other words, control device 100 determines whether to control the one of the devices to decrease the removal capability (Step S101). When control device 100 controls the one of the devices to decrease the removal capability (Yes in Step S101), control device 100 controls the other of the devices to compensate for the decreased removal capability of the one of the devices by increasing the removal capability of the other of the devices (Step S102). Subsequently, control device 100 determines whether to terminate the first mode or not (Step S103). When control device 100 does not control the one of the devices to decrease the removal capability (No in Step S101), control device 100 skips Step S102 and performs Step S103.

Although a termination condition for terminating the first mode varies depending on the control algorithm implemented in control system 500, for example, performing a process of changing the removal capability a predetermined number of times, continuing the first mode for a predetermined period of time, or receiving an input related to mode switching through a control panel is taken as an example of the condition.

When the termination condition is not satisfied and control device 100 does not terminate the first mode (No in Step S103), the processing returns to Step S101 and control device 100 continues the first mode. On the other hand, when the termination condition is satisfied and control device 100 terminates the first mode (Yes in Step S103), control device 100 controls ventilation device 110 and supply device 120 to switch to the second mode (Step S104). In the second mode, as described above, for example, uni-control is performed such that each of the devices operates at a constant removal capability or either one of the devices operates at a constant removal capability.

Subsequently, control device 100 determines whether to terminate the second mode or not (Step S105). Although a termination condition for terminating the second mode varies depending on the control algorithm implemented in control system 500, for example, continuing the second mode for a predetermined period of time, or receiving an input related to mode switching through a control panel is taken as an example of the condition.

When the termination condition is not satisfied and control device 100 does not terminate the second mode (No in Step S105), control device 100 repeats Step S105 and continues the second mode until the termination condition is satisfied. On the other hand, when the termination condition is satisfied and control device 100 terminates the second mode (Yes in Step S105), control device 100 controls ventilation device 110 and supply device 120 to switch to the first mode (Step S106). Subsequently, the processing returns to Step S101 and control device 100 repeats the above steps of the first mode again.

As shown in FIG. 33, the exemplary operation including the second operation is different from the above exemplary operation including the first operation in that the increase and decrease in the removal capability are reversed between the first mode and the second mode, More specifically, instead of Step S101 in FIG. 3A, control device 100 according to the present exemplary operation determines whether to control one of the devices to increase the removal capability (Step S201). When control device 100 according to the present exemplary operation controls the one of the devices to increase the removal capability (Yes in Step S201), control device 100 according to the present exemplary operation controls the other of the devices to cut (save) the amount of removal capability corresponding to the increase in the removal capability of the one of the devices by decreasing the removal capability of the other of the devices (Steps S202).

As described above, the present embodiment switches between, for example, the first mode in which the first action including Steps S101 to S103 is performed and, for example, the second mode in which the second action including Step S105 is performed. With this, each mode is selectively performed to appropriately control each of the devices.

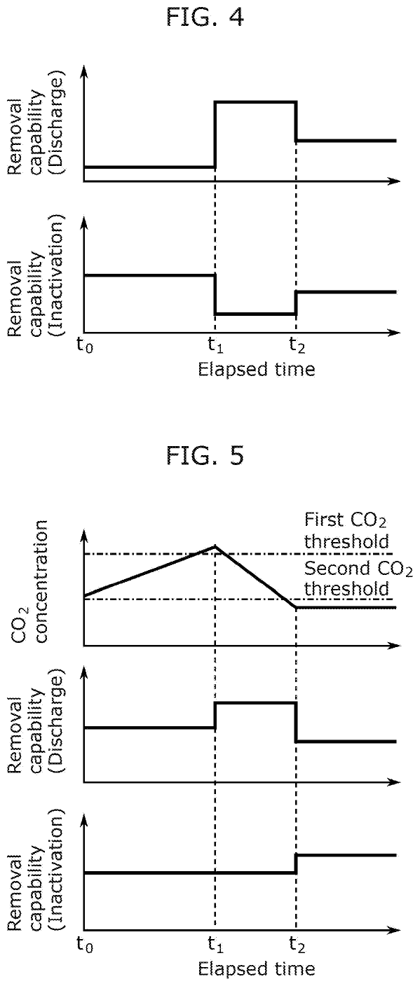

Next, more detailed exemplary operations each including specific details of the control algorithm are described below with reference to FIG. 4 through FIG. 10. FIG. 4 is a diagram illustrating the first specific operation according to the present embodiment. FIG. 4 shows the change in removal capability with respect to time for each of the devices. In the example shown in FIG. 4, the control mode of each device is switched at elapsed time t₁ and elapsed time t₂. More specifically, at elapsed time t₁, the removal capability of ventilation device 110 is increased. In response to this, the removal capability of supply device 120 is decreased in accordance with the second operation. For example, at elapsed time t₁, a predetermined time of a day triggers control device 100 to increase the ventilation air volume of ventilation device 110. With this, one or more ventilations are performed during a day, and the infectious material is removed.

During the period from elapsed time t₁ to elapsed time t₂, the above control mode continues, and at elapsed time t₂, the removal capability of supply device 120 is increased. In response to this, the removal capability of ventilation device 110 is decreased in accordance with the first operation. For example, control device 100 continues the ventilation-device 110 dominated removal of the infectious material during a predetermined period (from t₁ to t₂), and subsequently performs the supply-device 120 dominated removal of the infectious material. In doing so, the ventilation air volume of ventilation device 110 is decreased. With this, it is possible to spray the inactivation agent using supply device 120 while decreasing discharge outside the system by the ventilation. In other words, in this example, the first mode including the first operation is continuously performed. Note that FIG. 4 shows that the inactivation-removal capability is higher than the discharge-removal capability during the period from elapsed time t₀ to elapsed time t₁. This is because, for example, the inactivation-removal capability has been increased before elapsed time t₁ by manual operation such as user operation of the control panel by person 99. Accordingly, the second mode in which each device is controlled independently is performed before elapsed time

As described above, the order of performing the first mode and the second mode is not limited to the examples described in FIG. 3A and FIG. 3B. For example, the first mode may be performed after the second mode,

In the present example, at elapsed time t₁, the control of supply device 120 is changed in response to the change in control of ventilation device 110, as shown in the following equation (10):

$$\begin{matrix}[ Math . 10 ] & \\ {β}_{1} = {e}^{\frac{{Q}_{1} - {Q}_{t}}{V}} & ( 10 )\end{matrix}$$

where β₁ denotes the residual rate of the infectious material per unit time after the change at elapsed time t₁, and Q₁ denotes the ventilation air volume [m³/h] after the change at elapsed time t₁.

In the present example, at elapsed time t₂, the control of ventilation device 110 is changed in response to the change in control of supply device 120, as shown in the following equation (11):

[Math. 11]

Q₂=Qₜ+V lnβ₂ (11)

where β₂ denotes the residual rate of the infectious material per unit time after the change at elapsed time t₂, and Q₂ denotes the ventilation air volume [m³/h] after the change at elapsed time t₂,

FIG. 5 is a diagram illustrating the second specific operation according to the present embodiment. In addition to the figures similar to those in FIG. 4, FIG. 5 shows the change in CO₂ concentration obtained from CO₂ sensor 141 with respect to time. The present example is described as a case in which the ventilation air volume of ventilation device 110 is changed depending on the CO₂ concentration in the air, and in response to this, the inactivation agent is sprayed by supply device 120.

As shown in FIG. 5, in the present example, ventilation device 110 is controlled to keep the CO₂ concentration at an appropriate numerical value. For example, when indoor space 98 is used as a conference room or the like, the CO₂ concentration of approximately 1000 ppm or less is considered better as an appropriate CO₂ concentration. In view of this, in this exemplary operation, the CO₂ concentration measured by CO₂ sensor 141 is controlled to be lower than a CO₂ threshold basing the above 1000 ppm. In this case, the system according to the present example operates such that the performance of simultaneously controlled supply device 120 is kept at least at a certain level according to the above equations (8) and (9).

For example, each device is controlled in the second mode while the CO₂ concentration is lower than the first CO₂ threshold set to 1000 ppm or the like (until elapsed time t₁). Here, when the CO₂ concentration exceeds the first CO₂ threshold (at elapsed time control device 100 increases the ventilation air volume of ventilation device 110. In the period after elapsed time t₁, the CO₂ concentration in indoor space 98 switches from increasing to decreasing due to the ventilation. When the CO₂ concentration falls below the second CO₂ threshold which is sufficiently lower than the first CO₂ threshold and set to the CO₂ concentration of 600 ppm or the like, control device 100 decreases the ventilation air volume of ventilation device 110.

In accordance with the above operation, the removal capability of ventilation device 110 which has been set to a given level during the period from elapsed time t₀ to elapsed time t₁ is higher than the given level during the period from elapsed time t₁ to elapsed time t₂, and lower than the removal capability at elapse time t₂ during the period after elapsed time t₂. In the present example, the removal capability of supply device 120 does not change during the period from elapsed time t₁ to elapsed time t₂, but the removal capability may be decreased in terms of energy saving.

In the period after elapsed time t₂, two patterns of control are selectively performed based on the removal capability of ventilation device 110. In one of the two patterns, when the removal capability of ventilation device 110 after elapsed time t₂ is lower than the removal capability during the period from elapsed time t₀ to elapsed time t₁, the removal capability of supply device 120 is increased to compensate for the decrease in the removal capability of ventilation device 110, as shown in FIG. 5. In the other of the two patterns, when the removal capability of ventilation device 110 after elapsed time t₂ is higher than or equal to the removal capability during the period from elapsed time t₀ to elapsed time t₁, the removal capability of supply device 120 is maintained (or may be decreased).

In other words, each device described here is controlled according to the following equations (12) and (13).

$$\begin{matrix}[ Math . 12 ] & \\ \begin{matrix}{β}_{2} = {e}^{\frac{{Q}_{2} - {Q}_{t}}{V}} & ( {Q}_{2} < {Q}_{0} )\end{matrix} & ( 12 )\end{matrix}$$

$$\begin{matrix}[ Math . 13 ] & \\ \begin{matrix}{β}_{2} = {β}_{0} & ( {Q}_{2} \geq {Q}_{0} )\end{matrix} & ( 13 )\end{matrix}$$

Note that the upper limit of the removal capability of ventilation device 110 is determined by the maximum value of the ventilation air volume of ventilation device 110. In other words, in order to implement the total removal capability, it is necessary to take into account the maximum value and the minimum value of the removal capability of each of ventilation device 110 and supply device 120, The maximum value of the removal capability of ventilation device 110 is associated with the minimum value of the removal capability of supply device 120. In view of the above equation (8), the term of Q/V is maximized when the term of −lnβ is minimized. In this equation, V is the positive constant value, and thus Q is maximized when the term of Q/V is maximized, β is within the range of 0<β<1 from its nature. Accordingly, −lnβ is minimized when β is maximized. In other words, β is maximum value βₘₐₓ when Q is maximum value Qₘₐₓ. Accordingly, the following equation (14) is true,

[Math. 14]

Qₘₐₓ=Qₜ+V lnβₘₐₓ (14)

As with ventilation device 110, the upper limit of the removal capability of supply device 120 is determined by the maximum value of the volume of supplied inactivation agent of supply device 120. As with the above, considering the maximum value and the minimum value of the removal capability of each of ventilation device 110 and supply device 120, the maximum value of the removal capability of supply device 120 is associated with the minimum value of the removal capability of ventilation device 110. In view of the above equation (8), the term of Q/V is minimized when the term of −lnβ is maximized. In the same manner as the above, Q is minimized when the term of Q/V is minimized. In the range of 0<β<1, −lnβ is maximized when β is minimized. In other words, β is minimum value βₘᵢₙ when Q is minimum value Qₘᵢₙ. Accordingly, the following equation (15) is obtained,

[Math. 15]

Qₘᵢₙ=Qₜ+V lnβₘᵢₙ (15)

Moreover, the infection probability in transmission of the infectious material to person 99 can be estimated from the numerical value such as the CO₂ concentration in indoor space 98. When the CO₂ threshold is determined based on this estimation, the estimated infection probability can be kept low at a certain level. More specifically, the following equation (16) disclosed in NPL 1 is applied to the present disclosure:

$$\begin{matrix}[ Math . 16 ] & \\ P = 1 - {e}^{- \frac{Iqt ( {C}_{g} - {C}_{go} )}{{C}_{a} n}} & ( 16 )\end{matrix}$$

where P denotes the infection probability estimated based on the CO₂ concentration, I denotes the number of persons infected with the infectious material, q denotes the new emission rate of the infectious material per unit time [/h] such as a virus multiplication rate, C_{g} denotes the CO₂ concentration [ppm] in indoor space 98, C_{go} denotes the CO₂ concentration [ppm] in outdoor space 97, Cₐ denotes the ratio of CO₂ volume to the breathing volume of person 99, and n denotes the number of persons 99 present in indoor space 98. In the above equation (16), the elapsed time denoted by t can be regarded as the length of person's 99 stay in indoor space 98 in which the infectious material is floating. In other words, the elapsed time denoted by t can be treated as an exposure period of person 99 to the infectious material.

When the above equation (16) is rearranged with respect to C_{g}, the following equation (17) is obtained.

$$\begin{matrix}[ Math . 17 ] & \\ {C}_{g} = {C}_{go} - {C}_{a} \frac{( 1 - P )}{Iqt} & ( 17 )\end{matrix}$$

Here, FIG. 6 is a diagram illustrating the multiplication rates of the typical viruses. FIG. 6 shows the names of typical virus infections and the multiplication rates of viruses related to the infections in association with each other.

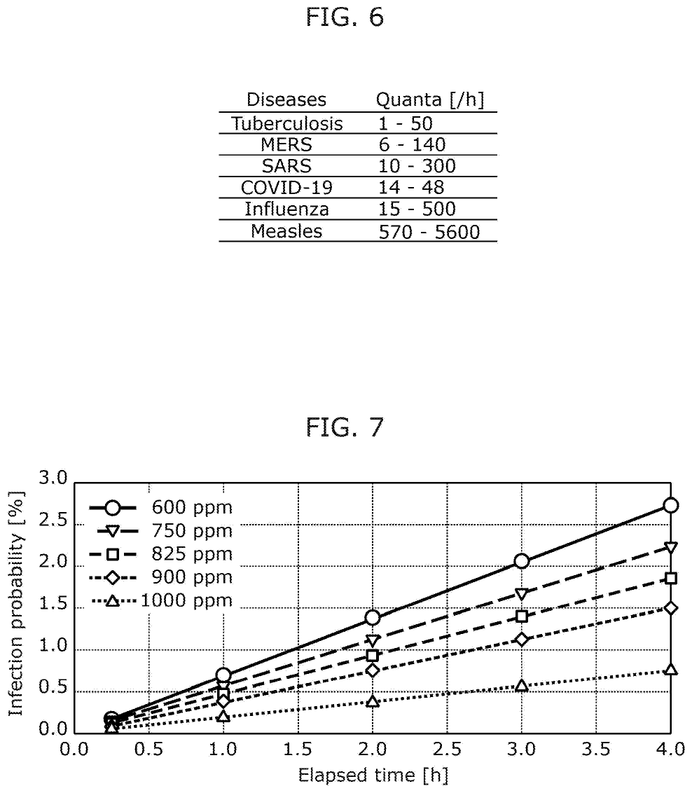

For example, the report about “SARS-CoV-2” related to “COVID-19”, which is an infection rapidly spread around the world from the end of 2019, shows that its multiplication rate is 14 through 48 per hour (see NPL 2). FIG. 7 is the first graph illustrating a transition of the infection probability with respect to the elapsed time. As one example, FIG. 7 shows a result of calculating the relationship between the elapsed time and the infection probability in indoor space 98 in which eight persons 99 including one person infected with SARS-CoV-2 are present. For example, when indoor space 98 is used for one hour under the above condition, the CO₂ threshold may be set to 825 ppm to keep the infection probability at a level of 0.5% or less.

Next, a direct control to keep the infection probability low without using the above CO₂ threshold is described. Here, the following equation (18) disclosed in NPL 3 is applied to the present disclosure:

$$\begin{matrix}[ Math . 18 ] & \\ P = 1 - {e}^{- \frac{Iqpt}{Q}} & ( 18 )\end{matrix}$$

where p denotes the breathing volume of person 99. In the above equation (18), the elapsed time denoted by t can be regarded as the length of person's 99 stay in indoor space 98 in which the infectious material is floating. In other words, the elapsed time denoted by t can be treated as an exposure period of person 99 to the infectious material.

When the above equation (18) is rearranged with respect to Q, the following equation (19) is obtained.

$$\begin{matrix}[ Math . 19 ] & \\ Q = - \frac{Iqpt}{ln ( 1 - P )} & ( 19 )\end{matrix}$$

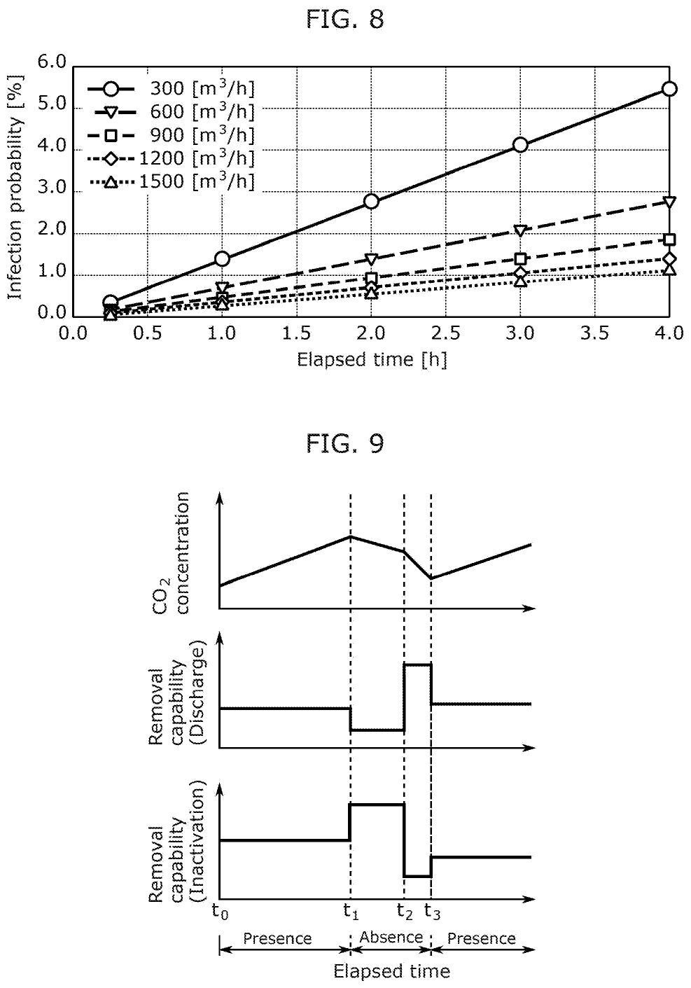

FIG. 8 is the second graph illustrating a transition of the infection probability with respect to the elapsed time. As with the case of FIG. 7, as one example, FIG. 8 shows a result of calculating the relationship between the elapsed time and the infection probability in indoor space in which persons 99 including one person infected with SARS-CoV-2 are present. Note that the calculation of the relationship between the elapsed time and the infection probability of FIG. 8 is performed under, for example, the assumption that indoor space 98 (in this case, a conference room or the like) is intended for use in a conference or the like during which persons 99 spend quiet times. Accordingly, p described here employs 0.3 [m³/h] as a typical breathing volume of each person 99 at rest.

For example, when indoor space 98 is used for one hour under the above condition, it is found that the ventilation air volume less than 600 [m³/h] is not adequate and the ventilation air volume of 900 [m³/h] or more is adequate to keep the infection probability at a level of 0.5% or less. Accordingly, when ventilation device 110 is controlled with the ventilation air volume of 900 [m³/h] or more, it is possible to keep the infection probability at a level of 0.5% or less in use for one hour.

Ventilation device 110 changes the ventilation air volume to a value greater than or equal to the threshold determined depending on the infection probability estimated in advance. After this, for example, ventilation device 110 is controlled to keep the ventilation air volume constant. In doing so, supply device 120 changes the volume of supplied inactivation agent in response to the performance of ventilation device 110, After this, for example, supply device 120 is controlled to keep the volume of supplied inactivation agent constant.

The foregoing configuration that changes the control of ventilation device 110 and the control of supply device 120 every time the CO₂ concentration is measured has the effect of always obtaining the optimal effectiveness in the removal of the infectious material since adjustment is performed every time according to the state of indoor space 98. However, changing the controls every time results in increase in the amount of calculation, and thus the calculation cost such as processing power or equipment required for the calculation is bloated.

In contrast, in the operation of control system 500 described here, when information regarding the number of users and a period of use of indoor space 98 can be obtained in advance, complicated calculation is not needed after the infection probability is determined once. In other words, the calculation cost can be reduced, and thus it is possible to efficiently prevent transmission of the infectious material. These control patterns in the trade-off relationship may be randomly changed by an administrator of control system 500, or may be automatically changed by monitoring the usage state of indoor space 98,

For example, when the number of persons in indoor space 98 detected by a human detecting sensor or the like is equal to the number of users on the schedule, the latter low-cost calculation is performed. Otherwise, the former optimal removal of the infectious material may be performed. Moreover, when the intended use of indoor space 98 is known in advance, ventilation device 110 and supply device 120 may be set to perform the controls according to the intended use.

Furthermore, βₘᵢₙ described in the above equation (15) depends on the maximum removal capability. The maximum removal capability varies depending on whether person 99 is present in indoor space 98. In other words, in a state in which person 99 is present in indoor space 98, a large volume of the inactivation agent that may affect person 99 cannot be sprayed. This results in large βₘᵢₙ. In contrast, in a state in which person 99 is absent in indoor space 98, the inactivation agent can be sprayed up to the capability limit of supply device 120. Accordingly, βₘᵢₙ can be applied as small as possible.

In view of this, in the exemplary operations described here, presence information indicating whether person 99 is present in indoor space 98 is obtained, and in the state in which person 99 is absent in indoor space 98, a higher concentration of the inactivation agent is sprayed.

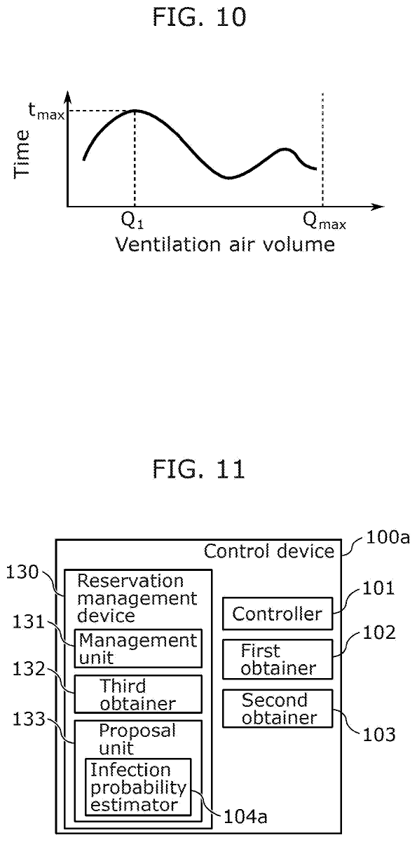

FIG. 9 is a diagram illustrating the third specific operation according to the present embodiment. As with the case of FIG. 5, FIG. 9 shows the change in removal capability with respect to time for each of the devices and the change in CO₂ concentration obtained from CO₂ sensor 141 with respect to time. FIG. 10 is a graph illustrating the relationship between the elapsed time and the ventilation air volume.

As shown in FIG. 9, in the present example, a transition from a state in which person 99 is present to a state in which person 99 is absent is used as a trigger to increase the volume of supplied inactivation agent, thereby enhancing the removal capability of supply device 120. The presence or absence of person 99 is determined based on the presence information obtained from presence sensor 142 as described above. In this case, the volume of supplied inactivation agent may base βₘᵢₙ in the state in which person 99 is absent, as described above. With this, while more effective inactivation removal is achieved, the effect on person 99 is kept low. Note that the volume of supplied inactivation agent is reduced before person 99 newly enters indoor space 98.

For example, while the volume of supplied inactivation agent is increased, control device 100 locks indoor space 98 to prevent person 99 from entering indoor space 98 by cooperating with a locking device of the doors and windows for use in entering and leaving indoor space 98, Moreover, in the present example, control device 100 obtains the next start time of use of indoor space 98 by accessing a schedule management server or the like, and reduces the volume of supplied inactivation agent according to the schedule.

In doing so, in view of the effect on person 99 from the remaining inactivation agent, the ventilation air volume of ventilation device 110 is increased before the start of the next use of indoor space 98 such that, for example, the remaining inactivation agent is removed to a level at which person 99 is not damaged or a level at which a feeling of strangeness regarding odor or like is not given to person 99. Simultaneously, this ventilation also decreases the CO₂ concentration in indoor space 98 to a predetermined level (e.g., the same level as outdoor space 97). In doing so, in order to achieve both the objects, a larger one of the ventilation air volumes required for the respective objects may be selected. In doing so, for example, in FIG. 9, t₂ denoting a time of decreasing the volume of supplied inactivation agent and increasing the ventilation air volume is determined by back calculation from t₃ denoting a start time of the next use of indoor space 98. Here, the following equations (20) and (21) are used.

$$\begin{matrix}[ Math . 20 ] & \\ {C}_{g} ( {t}_{2} ) = {C}_{go} + ( {C}_{g} ( {t}_{1} ) - {C}_{go} ) {e}^{- \frac{{Q}_{1}}{V} ( {t}_{2} - {t}_{1} )} & ( 20 )\end{matrix}$$

$$\begin{matrix}[ Math . 21 ] & \\ {C}_{g} ( {t}_{3} ) = {C}_{go} + ( {C}_{g} ( {t}_{2} ) - {C}_{go} ) {e}^{- \frac{{Q}_{2}}{V} ( {t}_{3} - {t}_{2} )} & ( 21 )\end{matrix}$$

Note that, in the above equation (20), C_{g}(t₂) denotes the CO₂ concentration [ppm] in indoor space 98 at elapsed time t₂, C_{go} denotes the CO₂ concentration [ppm] in outdoor space 97, C_{g}(t₁) denotes the CO₂ concentration [ppm] in indoor space 98 at elapsed time t₁, and Q₁ denotes the ventilation air volume [m³/h] during the period from elapsed time t₁ to elapsed time t₂. In the above equation (21), C_{g}(t₃) denotes the CO₂ concentration [ppm] in indoor space 98 at elapsed time t₃, and Q₂ denotes the ventilation air volume [m³/h] during the period from elapsed time t₂ to elapsed time t₃.

Here, in order to discharge the CO₂ and the remaining inactivation agent in a short period, the maximum ventilation air volume may be applied to the ventilation air volume of the period from elapsed time t₂ to elapsed time t₃ (i.e., Q₂=Qₘₐₓ). For example, in order to prevent uneven effect caused by air turbulence and discharge of the inactivation agent to the outside due to the ventilation, in the period for which the inactivation agent is aggressively sprayed (here, from elapsed time t₁ to elapsed time t₂), ventilation device 110 may be stopped or a large volume of the inactivation agent may be sprayed. In the example described here, the former is performed.

In this case, Q₁=0 is satisfied. Accordingly, when the above equations (20) and (21) are rearranged with respect to t₂, the following equation (22) is obtained.

$$\begin{matrix}[ Math . 22 ] & \\ {t}_{2} = {t}_{3} - \frac{V}{{Q}_{max}} ln ( \frac{{C}_{g} ( {t}_{3} ) - {C}_{go}}{{C}_{g} ( {t}_{1} ) - {C}_{go}} ) & ( 22 )\end{matrix}$$

In contrast, for example, when Q₁>0 is satisfied, the ventilation aft volume selected to maximize the elapsed time by reference to the graph shown in FIG. 10 may be employed as With this, the spraying period of the inactivation agent can be set as long as possible, and thus it is possible to enjoy the effective inactivation removal to the maximum.

Note that the above times t₁ and t₃ may be provided by input from person 99, In other words, t₁ may be determined by operating “use end button” or the like displayed on the control panel. Also, t₃ may be determined by operating “use start button” or the like. In doing so, for example, the control panel may be also placed in outdoor space 97 and configured to be able to set t₃ without entering indoor space 98 filled with the inactivation agent. Moreover, as described in the above, control system 500 may cooperate with a system for managing the use schedule of indoor space 98 by making a reservation. This system for managing the schedule or the like is described below in details.

(Reservation Management System for Use of Indoor Space)

FIG. 11 is a block diagram illustrating the functional configuration of the control device including a reservation management device according to the embodiment. FIG. 11 shows only control device 100a of control system 500. However, as described above, control device 100a is connected with ventilation device 110 and supply device 120 and used to control these devices.

In this example, the description of the configurations of controller 101, first obtainer 102, and second obtainer 103 in control device 100a is omitted since they are the same as those in control device 100 described above. Control device 100a differs from control device 100 described above in that control device 100a includes reservation management device 130. Accordingly, the description is focused on this.

Reservation management device 130 manages the use schedule of indoor space 98 using a reservation made by person 99 (hereinafter, also referred to as a user who uses indoor space 98), and is implemented by causing a processor and a memory to execute a predetermined program. Reservation management device 130 includes management unit 131, third obtainer 132, and proposal unit 133.

Here, proposal unit 133 includes infection probability estimator 104a corresponding to infection probability estimator 104 in control device 100 described above. In other words, in this example, the function of infection probability estimator 104 in control device 100 is implemented by infection probability estimator 104a included in proposal unit 133. In other words, infection probability estimator 104a is shared between control device 100a and reservation management device 130. Note that infection probability estimator 104a is not necessarily configured to be shared. The infection probability estimator for control device 100a and infection probability estimator 104a for reservation management device 130 may be separately provided. When the infection probability estimator is provided independently, reservation management device 130 may be implemented as an independent device instead of being included in control device 100a. For example, information terminal such as a smart phone belonging to a user may be used as reservation management device 130.

Management unit 131 is a database that integrally manages reservation information for a user to use indoor space 98. Management unit 131 is implemented by a controller and a storage not shown in FIG. 11. As one example, based on start time of use and end time of use indicated in reservation information inputted by a user, the period of use is managed without overlapping with another period of use with respect to time. For example, obtainment of the reservation information may be implemented by user's operation of the control panel of control device 100a. Alternatively, the reservation information inputted via an information terminal such as a smart phone may be obtained through a network.

Management unit 131 presents the reservation information under management in response to a request from a user. The user inputs a new reservation into an available time slot while referring to the presented reservation information. This prevents double booking of indoor space 98 and facilitates sharing the reservation information between users or user groups,

The reservation management system for use of indoor space according to the present embodiment further includes third obtainer 132 and proposal unit 133. With this, at the completed stage of user's input of a reservation, it is possible to estimate the infection probability under the use of indoor space 98 when reserved, and propose a condition of use under which the infection probability is more reduced.

Third obtainer 132 is a functional unit that obtains user information regarding a user included in the reservation information. As with the case of management unit 131, third obtainer 132 may extract the user information by directly obtaining the reservation information. Alternatively, third obtainer 132 may obtain only the extracted user information among the reservation information obtained by management unit 131. As described above, third obtainer 132 is implemented as a communication module for obtaining the user information.

The user information includes the number of users of indoor space 98, a period of use of indoor space 98, an intended use of indoor space 98, and the like.

Proposal unit 133 is a processing unit that calculates an infection probability based on the obtained user information and proposes a condition of use under which the infection probability is reduced. Proposal unit 133 is implemented by causing a processor and a memory to execute a predetermined program. First, proposal unit 133 calculates the infection probability in transmission of the infectious material to the user, which is estimated using infection probability estimator 104a under the use of indoor space 98 according to the details of the user information. The calculated infection probability is compared with a reference infection probability to determine whether a proposal is needed. More specifically, the reference infection probability is an upper limit of the infection probability. The infection probability is recommended to be lower than the upper limit. Hereinafter, the upper limit of the infection probability is also referred to as an upper infection probability limit. When the calculated infection probability exceeds the upper infection probability limit, proposal unit 133 proposes a condition of use under which the infection probability falls below the upper infection probability limit.

As described above, the reservation management system for use of indoor space according to the present embodiment further proposes a condition of use based on the user information, and thus it is possible to offer users indoor space 98 in which the infection probability is appropriately managed. The above reservation management system for use of indoor space is one example of the proposal system.

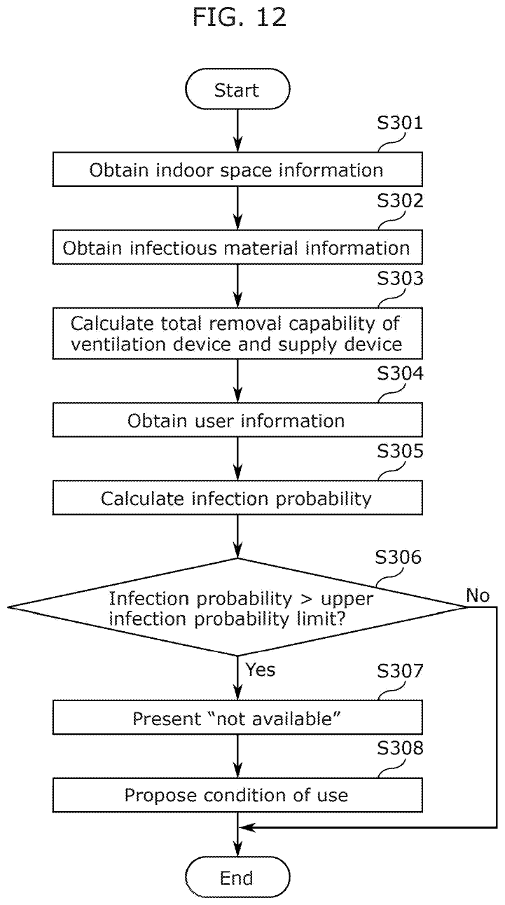

The operation of the reservation management system for use of indoor space is described below with reference to FIG. 12. FIG. 12 is a flow chart illustrating the operation related to a proposal of a condition of use, of the reservation management system for use of indoor space according to the present embodiment. As shown in FIG. 12, first, proposal unit 133 obtains various kinds of information necessary for the calculation of the infection probability. More specifically, proposal unit 133 obtains indoor space information (Step S301). The indoor space information is related to the state of indoor space 98, and includes parameters contributing to the calculation of the infection probability. More specifically, the indoor space information includes parameters such as the design size of indoor space 98, the ventilation air volume of ventilation device 110 placed in indoor space 98, and the volume of inactivation agent supplied from supply device 120 placed in indoor space 98.

Note that the indoor space information may include information regarding the placement status of ventilation device 110 and supply device 120 in indoor space 98. In other words, the case in which at least one of ventilation device 110 or supply device 120 is not placed in indoor space 98 is possible. In this case, for example, an effective ventilation air volume may be calculated using the change in CO₂ concentration measured by CO₂ sensor 141 or the like during an available time slot of indoor space 98. The effective ventilation air volume is calculated using the following equation (23):

$$\begin{matrix}[ Math . 23 ] & \\ {Q}_{e} = \frac{V}{T} ln ( \frac{{C}_{gs} - {C}_{go}}{{C}_{ge} - {C}_{go}} ) & ( 23 )\end{matrix}$$

where Qₑ denotes the effective ventilation air volume [m³/h], T denotes the elapsed time [h] from the time when the indoor space becomes available, C_{gs} denotes the CO₂ concentration [ppm] at the time when the indoor space becomes available, and C_{ge} denotes the CO₂ concentration [ppm] at elapsed time T from the time when the indoor space becomes available. The effective ventilation air volume described here corresponds to Q in the above equation (9), and thus the following equation (24) is true.

[Math. 24]

Qₜ=Qₑ−V lnβ (24)

Proposal unit 133 also obtains infectious material information regarding the infectious material which is an estimation target of the infection probability (Step S302). Parameters specific to the infectious material are obtained from, for example, a database. Accordingly, the infectious material information includes information for identifying the infectious material, a growth rate per unit time and an upper infection probability limit obtained by referring to the database in the identifying, the number of persons infected with the infectious material (infection numbers), etc.

Proposal unit 133 calculates the total removal capability of ventilation device 110 and supply device 120 using the above equation (8) based on the various kinds of information obtained in Step S301 and Step S302 (Step S303). Note that, in the above operation, the numerical values are repeatedly available unless indoor space 98 and the infectious material are changed, and thus the obtained and calculated numerical values may be stored in a storage or the like. In the subsequent operations, each operation can be started from the following step S304 by referring to the storage.

Next, proposal unit 133 obtains the user information (Step S304). Proposal unit 133 calculates the infection probability under the use of indoor space 98, based on the obtained user information and the various kinds of information obtained in Step S301 and Step S302 (Step S305).

The infection probability described here may be calculated using the above equation (16) or the above equation (18). In the case of using the above equation (16), a difference in CO₂ concentration between indoor space and outdoor space with respect to the ratio of CO₂ volume to the breathing volume of a user, denoted by (C_{gt}−C_{go})/Cₐ, is needed. This value can be calculated using the following equation (25):

$$\begin{matrix}[ Math . 25 ] & \\ {f}_{t} = \frac{{C}_{gt} - {C}_{go}}{{C}_{a}} = \frac{1}{{C}_{a}} \{ 1 - \frac{V}{{Q}_{t} T} ( 1 - {e}^{- \frac{{Q}_{t} T}{V}} ) \} & ( 25 )\end{matrix}$$

where fₜ denotes the difference in CO₂ concentration between indoor space and outdoor space with respect to the ratio of CO₂ volume to the breathing volume of a user, and C_{gt} denotes the CO₂ concentration in indoor space 98 at elapsed time T.

Back to FIG. 12, proposal unit 133 compares the calculated infection probability with the upper infection probability limit set for each of the types of infectious material, to determine whether the infection probability exceeds the upper infection probability limit (Step S306). When it is determined that the infection probability does not exceed the upper infection probability limit (No in Step S306), proposal unit 133 terminates the processing. On the other hand, when it is determined that the infection probability exceeds the upper infection probability limit (Yes in Step S306), proposal unit 133 presents “not available” indicating that indoor space 98 is not available for the intended use when reserved (Step S307).

For example, this may be implemented by sending a push notification to the information terminal used for a user to make a reservation or displaying “not available” on the display screen of a control terminal. The presentation described here may be displayed as an image with a combination of characters, shapes, symbols, etc. Alternatively, a sound meaning “not available” may be reproduced from a speaker or the like,

Subsequently, proposal unit 133 proposes a condition of use of indoor space 98 that reduces the infection probability to below the upper infection probability limit (Step S308).

A proposal of the condition of use by proposal unit 133 is listed below according to its type.

First, proposal unit 133 suppresses the increase in infection probability during use of indoor space 98 by shorten the period of the use. For example, when the infection probability is calculated based on the above equation (16), a proposed period of use is determined based on the following equation (26):

$$\begin{matrix}[ Math . 26 ] & \\ {t}_{p} = - \frac{n}{{f}_{t} Iq} ln ( 1 - {P}_{t} ) & ( 26 )\end{matrix}$$

where tₚ denotes the proposed period of use, and Pₜ denotes the infection probability when the proposed period of use is employed,

Alternatively, for example, when the infection probability is calculated based on the above equation (18), the proposed period of use is determined based on the following equation (27):

$$\begin{matrix}[ Math . 27 ] & \\ {t}_{p} = - \frac{{Q}_{t}}{Iqp} ln ( 1 - {P}_{t} ) & ( 27 )\end{matrix}$$

Moreover, proposal unit 133 suppresses the increase in infection probability during use of indoor space 98 by changing the intended use of the use. For example, it is known that the CO₂ volume when the user's activity in indoor space 98 is normal sport is about 5 times as large as the CO₂ volume when the user's activity in indoor space 98 is general office work. This arises from an increase in breathing volume of the user, and the increase in breathing volume causes an increase in infection probability. In view of this, proposal unit 133 proposes a condition of use such that the intended use scheduled by a user is changed to another intended use in which the breathing volume is lower than that of the intended use scheduled by the user.

Moreover, proposal unit 133 suppresses the increase in infection probability during use of indoor space 98 by increasing the performance power of supply device 120 in indoor space 98 (i.e., the volume of sprayed inactivation agent is increased). For example, when the infection probability is calculated based on the above equation (16), a proposed volume of supplied inactivation agent is determined based on the following equation (28):

$$\begin{matrix}[ Math . 28 ] & \\ {f}_{t} = \frac{1}{{C}_{a}} \{ 1 - \frac{V}{{Q}_{p} T} ( 1 - {e}^{- \frac{{Q}_{p} T}{V}} ) \} & ( 28 )\end{matrix}$$

where Qₚ denotes the ventilation air volume when the proposed volume of supplied inactivation agent is employed. The approximate value of Qₚ is calculated by applying Maclaurin expansion to the above equation (28), and the residual rate of the infectious material per unit time when the proposed volume of supplied inactivation agent is employed is calculated using the calculated approximate value and the following equation (29).

$$\begin{matrix}[ Math . 29 ] & \\ {β}_{p} = {e}^{\frac{{Q}_{p} - Q}{V}} & ( 29 )\end{matrix}$$

where βₚ denotes the residual rate of the infectious material per unit time when the proposed volume of supplied inactivation agent is employed.

Alternatively, for example, when the infection probability is calculated based on the above equation (18), the proposed volume of supplied inactivation agent is determined based on the following equation (30).

$$\begin{matrix}[ Math . 30 ] & \\ {Q}_{p} = - \frac{Iqpt}{ln ( 1 - {P}_{t} )} & ( 30 )\end{matrix}$$

The residual rate of the infectious material per unit time when the proposed volume of supplied inactivation agent is employed is calculated using the value of Qₚ calculated here and the above equation (29). The proposal of the volume of supplied inactivation agent described here includes a proposal of changing the volume of supplied inactivation agent from 0 to a volume greater than 0. In other words, a proposal of changing the operational status of supply device 120 from “off” to “on” or a proposal of recommending that supply device 120 is newly placed in indoor space 98 when supply device 120 is not placed may be included.

Moreover, proposal unit 133 suppresses the increase in infection probability during use of indoor space 98 by increasing the performance power of ventilation device 110 in indoor space 98 (i.e., the ventilation air volume is increased). For example, when the infection probability is calculated based on the above equation (16), the proposed ventilation air volume is determined based on the above equation (28). In other words, the approximate value of Qₚ calculated by applying Maclaurin expansion to the above equation (28) is proposed.

Alternatively, for example, when the infection probability is calculated based on the above equation (18), the proposed ventilation air volume is determined based on the above equation (30). In other words, the value of Qₚ calculated using the above equation (30) is proposed.

Moreover, proposal unit 133 suppresses the increase in infection probability during use of indoor space 98 by lowering the target CO₂ concentration in ventilation by ventilation device 110 (i.e., decreasing the difference in CO₂ concentration between the indoor space and the outdoor space) to increase the ventilation air volume. For example, when the infection probability is calculated based on the above equation (16), a proposed difference in CO₂ concentration is determined based on the following equation (31).

$$\begin{matrix}[ Math . 31 ] & \\ {f}_{p} = - \frac{n}{tIq} ln ( 1 - {P}_{t} ) & ( 31 )\end{matrix}$$

The above equation (31) is substituted into the following equation (32):

[Math. 32]

C_{gp}−C_{go}=Cₐfₚ (32)

where C_{gp} denotes the CO₂ concentration in indoor space 98 for the proposed difference in CO₂ concentration.

Moreover, use of another indoor space 98 under an appropriate condition in which the infection probability is lower than the upper infection probability limit may be proposed instead of indoor space 98 scheduled to be used by a user. Note that only one of the conditions of use described above may be proposed, or a combination thereof may be proposed. Moreover, the above proposals are performed when a reservation for use of indoor space 98 is inputted, but in actual use, the proposal may be performed in real time based on the actual measured value.

### Advantageous Effects, Etc.

As described above, control system 500 according to the present embodiment includes: ventilation device 110 that replaces indoor-space 98 air containing an infectious material with outdoor-space 97 air to perform discharge removal of the infectious material; supply device 120 that supplies an inactivation agent for inactivating the infectious material to indoor space 98 to perform inactivation removal of the infectious material; and control device 100 that controls ventilation device 110 and supply device 120, in which control device 100 switches between a first mode and a second mode. The first mode is a mode in which at least one of the following is performed: a first operation that decreases a removal capability of one of ventilation device 110 and supply device 120 when a removal capability of an other of ventilation device 110 and supply device 120 is increased; or a second operation that increases the removal capability of the one of ventilation device 110 and supply device 120 when the removal capability of the other of ventilation device 110 and supply device 120 is decreased. The second mode is different from the first mode.

When the removal capability of one of ventilation device 110 and supply device 120 is decreased, such control system 500 can compensate for this decrease by increasing the removal capability of the other of ventilation device 110 and supply device 120. In contrast, when the removal capability of one of ventilation device 110 and supply device 120 is increased, the removal capability of the other of ventilation device 110 and supply device 120 is decreased, thereby preventing excessive removal capability while maintaining the minimum removal capability. Accordingly, the devices can compensate each other as need in terms of the removal capability, and the cost of the excessive removal capability can be eliminated. Accordingly, it is possible to more effectively prevent transmission of the infectious material to persons 99.

Moreover, for example, in the first mode, control device 100 may control ventilation device 110 and supply device 120 to increase a total removal capability for the infectious material to a removal threshold or greater. The total removal capability is defined by the above equation (8) where V denotes a size of indoor space 98, Q denotes a ventilation air volume of ventilation device 110 which is a replacement aft volume per unit time, and 3 denotes a residual rate of the infectious material remaining per unit time in the inactivation removal using the inactivation agent.

With this, the total removal capability of ventilation device 110 and supply device 120 is kept above the removal threshold. In other words, the total removal capability does not fall below the set removal threshold, and thus the removal capability can be more precisely specified. Accordingly, it is possible to more effectively prevent transmission of the infectious material to persons 99.

Moreover, for example, in the second mode, control device 100 may perform a third operation that keeps the removal capability of one of ventilation device 110 and supply device 120 constant when the removal capability of an other of ventilation device 110 and supply device 120 is increased.

With this, when one of the removal capabilities is increased, the other of the removal capabilities is not decreased, and thus the total removal capability can be increased. In other words, the removal effect can be enhanced, and thus it is possible to more effectively prevent transmission of the infectious material to persons 99.

Moreover, for example, in the first mode, control device 100 may: obtain a CO₂ concentration in indoor space 98 from CO₂ sensor 141 that measures the CO₂ concentration in indoor space 98; and control ventilation device 110 to replace air to reduce the CO₂ concentration obtained to a CO₂ threshold or less.

With this, even when ventilation device 110 independently performs the operation that reduces the CO₂ concentration, the total removal capability can be exerted by supply device 120 without excess or deficiency. In addition, this operation appropriately maintains the CO₂ concentration in indoor space 98, and thus indoor space 98 is maintained to be suitable for use more than ever. Accordingly, it is possible to more effectively prevent transmission of the infectious material to persons 99.

Moreover, for example, the CO₂ threshold may be a target CO₂ concentration in indoor space 98 that is determined by an exposure period during which person 99 present in indoor space 98 is exposed to the infectious material and an upper infection probability limit that is an upper limit of an infection probability in transmission of the infectious material to person 99.

With this, transmission of the infectious material to persons 99 can be prevented simply by managing the infection probability using the CO₂ concentration in indoor space 98 as an indicator and appropriately maintaining the CO₂ concentration. In addition, in such a management, the infection probability can be monitored in real time, and thus it is possible to take additional measures when the infection probability is temporarily increased or the like. Accordingly, it is possible to more effectively prevent transmission of the infectious material to persons 99.

In the first mode, control device 100 may: calculate a ventilation air volume threshold determined by an exposure period of person 99 present in indoor space 98 to the infectious material and an upper infection probability limit that is an upper limit of an infection probability in transmission of the infectious material to person 99; and control ventilation device 110 to replace a volume of air greater than or equal to the ventilation air volume threshold.

With this, ventilation device 110 is controlled with an adequate ventilation air volume based on the infection probability and the exposure period to the infectious material. Accordingly, it is possible to more appropriately and more effectively prevent transmission of the infectious material to persons 99.

Moreover, for example, control device 100 may: obtain presence information regarding whether person 99 is present in indoor space 98 from presence sensor 142 that detects whether person 99 is present in indoor space 98; and, using a transition from a state in which person 99 is present in indoor space 98 to a state in which person 99 is absent in indoor space 98 based on the obtained presence information as a trigger, start the first mode with the second operation, and continues the first mode with the first operation after a predetermined period of time.

With this, the first mode in which the first operation is appropriately combined with the second operation according to whether person 99 is present or not can be performed. For example, in indoor space 98 where persons 99 are absent, control device 100 causes supply device 120 to spray the inactivation agent in the first operation. In doing so, even when the removal effect by inactivation is enhanced by spraying the inactivation agent up to a high-concentration level that affects the human body, the safety is ensured since persons 99 are absent. Moreover, the subsequent operation of ventilation device 110 causes the first mode to be terminated after discharging the remaining inactivation agent through ventilation, and thus the effect on the persons from the inactivation agent is sufficiently reduced at a time when person 99 newly enters indoor space 98. Accordingly, it is possible to more effectively prevent transmission of the infectious material to persons 99.

Moreover, a control method according to the present embodiment is a control method of controlling a ventilation device and a supply device. The ventilation device replaces indoor-space air containing an infectious material with outdoor-space air to perform discharge removal of the infectious material. The supply device supplies an inactivation agent for inactivating the infectious material to an indoor space to perform inactivation removal of the infectious material. The control method includes: a first step of performing at least one of the following: a first operation that decreases a removal capability of the supply device when a removal capability of the ventilation device is increased; or a second operation that increases the removal capability of the supply device when the removal capability of the ventilation device is decreased; and a second step of performing an operation different from that of the first step.

With this, it is possible to produce the same effect as control system 500 described above.

Moreover, the above control method can be also implemented as a program for causing a computer to execute the above control method.

With this, it is possible to produce the same effect as the control method described above using a computer.

Moreover, the above embodiment may be implemented as a control device that is connected to ventilation device 110 and supply device 120 and switches between a first mode and a second mode. The first mode is a mode in which at least one of the following is performed: a first operation that decreases a removal capability of one of the ventilation device and the supply device when a removal capability of an other of the ventilation device and the supply device is increased; or a second operation that increases the removal capability of the one of the ventilation device and the supply device when the removal capability of the other of the ventilation device and the supply device is decreased. The second mode is different from the first mode.

With this, even a single control device can produce the same effect as the control system described above.

### Other Embodiments

The control system or the like according to the present disclosure has been described in accordance with the above embodiment, but the present disclosure is not limited to the above embodiment.

Moreover, in the above embodiments, a process performed by a specified processing unit may be performed by another processing unit. The processing order of processes may be changed, or processes may be performed in parallel. The allocation of components in the control system to the devices is one example. For example, a component in one of the devices may be included in the other device,

For example, the processes described in the above embodiments may be performed by a single device (system) as integrated processing, or by multiple devices as distributed processing. The processor that executes the above program may be singular or plural. In other words, the processor may perform the integrated processing or the distributed processing.

In the above embodiments, all or a part of the components such as a controller may be configured with dedicated hardware, or may be implemented by executing a software program suitable for each component. Each component may be implemented by a program executer such as a central processing unit (CPU) or a processor reading and executing a software program recorded on a recording medium such as a hard disk drive (HDD) or a semiconductor memory.

The component such as a controller may be configured in one or more electronic circuits. The one or more electronic circuits may be each a general-purpose circuit or a dedicated circuit.

The one or more electronic circuits may include, for example, a semiconductor device, an integrated circuit (IC), or a large scale integration (LSI) circuit. The IC or LSI circuit may be integrated into a single chip or multiple chips. Due to a difference in the degree of integration, the electronic circuit referred here to as an IC or LSI circuit may be referred to as a system LSI circuit, a very large scale integration (VLSI) circuit, or an ultra large scale integration (ULSI) circuit. A field programmable gate array (FPGA) which is programmable after manufacturing of the LSI circuit also can be used for the same purposes.

Moreover, these general and specific aspects of the present disclosure may be implemented using a system, a device, a method, an integrated circuit, or a computer program. Alternatively, these may be implemented using a non-transitory computer-readable recording medium such as an optical disk, HDD, or semiconductor memory storing the computer program. These also may be implemented using any combination of systems, devices, methods, integrated circuits, computer programs, or recording media,

The present disclosure may also include embodiments as a result of adding, to the embodiments, various modifications that may be conceived by those skilled in the art, and embodiments obtained by combining elements and functions in the embodiments in any manner without departing from the scope of the present invention.

### REFERENCE SIGNS LIST

97 outdoor space
98 indoor space
99 person
100, 100a control device
110 ventilation device
120 supply device
141 CO₂ sensor
142 presence sensor
500 control system

1. A control system, comprising:
a ventilation device that replaces indoor-space air containing an infectious material with outdoor-space air to perform discharge removal of the infectious material;
a supply device that supplies an inactivation agent for inactivating the infectious material to an indoor space to perform inactivation removal of the infectious material; and
a control device that controls the ventilation device and the supply device, wherein
the control device switches between a first mode and a second mode,
the first mode being a mode in which at least one of the following is performed: a first operation that decreases a removal capability of one of the ventilation device and the supply device when a removal capability of an other of the ventilation device and the supply device is increased; or a second operation that increases the removal capability of the one of the ventilation device and the supply device when the removal capability of the other of the ventilation device and the supply device is decreased,
the second mode being different from the first mode.

2. The control system according to claim 1, wherein
in the first mode,
the control device controls the ventilation device and the supply device to increase a total removal capability for the infectious material to a removal threshold or greater, the total removal capability being defined by:
$$\frac{Q}{V} - ln β \qquad [ Math . 1 ]$$
where V denotes a size of the indoor space, Q denotes a ventilation air volume of the ventilation device which is a replacement air volume per unit time, and β denotes a residual rate of the infectious material remaining per unit time in the inactivation removal using the inactivation agent.

3. The control system according to claim 1, wherein
in the second mode, the control device performs a third operation that keeps the removal capability of one of the ventilation device and the supply device constant when the removal capability of an other of the ventilation device and the supply device is increased.

4. The control system according to claim 1, wherein
in the first mode, the control device:
obtains a CO₂ concentration in the indoor space from a CO₂ sensor that measures the CO₂ concentration in the indoor space; and
controls the ventilation device to replace air to reduce the CO₂ concentration obtained to a CO₂ threshold or less.

5. The control system according to claim 4, wherein
the CO₂ threshold is a target CO₂ concentration in the indoor space that is determined by an exposure period during which a person present in the indoor space is exposed to the infectious material and an upper infection probability limit that is an upper limit of an infection probability in transmission of the infectious material to the person.

6. The control system according to claim 1, wherein
in the first mode, the control device:
calculates a ventilation air volume threshold determined by an exposure period of a person present in the indoor space to the infectious material and an upper infection probability limit that is an upper limit of an infection probability in transmission of the infectious material to the person; and
controls the ventilation device to replace a volume of air greater than or equal to the ventilation air volume threshold.

7. The control system according to claim 1, wherein
the control device:
obtains presence information regarding whether a person is present in the indoor space from a presence sensor that detects whether a person is present in the indoor space; and
using a transition from a state in which a person is present in the indoor space to a state in which a person is absent in the indoor space based on the obtained presence information as a trigger, starts the first mode with the second operation, and continues the first mode with the first operation after a predetermined period of time.

8. A control method of controlling a ventilation device and a supply device, the ventilation device replacing indoor-space air containing an infectious material with outdoor-space air to perform discharge removal of the infectious material, the supply device supplying an inactivation agent for inactivating the infectious material to an indoor space to perform inactivation removal of the infectious material, the control method comprising:
a first step of performing at least one of the following: a first operation that decreases a removal capability of the supply device when a removal capability of the ventilation device is increased; or a second operation that increases the removal capability of the supply device when the removal capability of the ventilation device is decreased; and
a second step of performing an operation different from that of the first step.

9. A non-transitory computer-readable recording medium for use in a computer, the recording medium having Aa program recorded thereon for causing the computer to execute the control method according to claim 8.
